(19) United States
(12) Patent Application Publication
(10) Pub. No.: US 2025/0025282 A1
(43) Pub. Date: Jan. 23, 2025

(54) OVAL AORTIC STENT

(71) Applicant: EDWARDS LIFESCIENCES CORPORATION, Irvine, CA (US)

(72) Inventors: Yuval Kasher, Kfar Shmuel (IL); Emil Karapetian, Huntington Beach, CA (US); Roei Mayo, Irvine, CA (US)

(21) Appl. No.: 18/910,265

(22) Filed: Oct. 9, 2024

Related U.S. Application Data
(63) Continuation of application No. PCT/US2023/018834, filed on Apr. 17, 2023.
(60) Provisional application No. 63/332,989, filed on Apr. 20, 2022.

(51) Int. Cl. A61F 2/06

(52) U.S. Cl. CPC A61F 2/06; A61F 2230/0008; A61F 2250/0039

(57) ABSTRACT

A stent comprises a network of elastically deformable struts having a biased cross-sectional shape and forming a first end portion having a first central diameter in a major dimension and a second central diameter in a minor dimension, wherein the first central diameter is greater than the second central diameter, a second end portion having the first central diameter in the major dimension and the second central diameter in the minor dimension, and a middle portion situated between the first end portion and the second end portion, the middle portion having a third central diameter in the major dimension and the second central diameter in the minor dimension, the third central diameter being less than the first central diameter.

### RELATED APPLICATION

This application is a continuation of International Patent Application No. PCT/US2023/018834, filed Apr. 17, 2023, which claims the benefit of U.S. Provisional Application No. 63/332,989, filed on Apr. 20, 2022, the disclosures of which are hereby incorporated by reference in their entireties.

### BACKGROUND

The present disclosure generally relates to devices and methods for vascular repair.

Catheter systems, such as treatment, delivery, and/or deployment catheters, can be used to treat patients internally. For example, delivery catheter systems can be used to deliver and deploy prosthetic devices, such as prosthetic heart valves, at locations inside the body. Prosthetic heart valves can be delivered to a treatment site (e.g., aortic, mitral, tricuspid, and/or pulmonary valve position) within a patient using transcatheter techniques.

### SUMMARY

Described herein are systems, devices, and methods for restoring compliance to non-compliant vascular structures by causing temporary changes in the cross-sectional geometry of a blood vessel without changing or with minimal change to a peripheral wall length of the blood vessel during initial implant. More specifically, the relaxed/diastolic cross-sectional shape of the blood vessel may be altered into a non-circular shape and/or, when subjected to increased pressure (e.g., during systole), the blood vessel may assume a circular (or more circular) shape. Because a highest area-to-periphery ratio may be achieved with a circular shape, a device which provides for transition between a non-circular shape and a circular (or more circular) shape may allow for cross-sectional area expansion. Accordingly, some examples herein may allow for expansion/contraction of the blood vessel cross-sectional area between diastole/systole without the need for high elasticity of the blood vessel walls.

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

It should be understood that each of the elements disclosed herein can be used with any and all of the elements disclosed herein, even though the specific combination of elements may not be explicitly shown in the figures herein. In other words, based on the explanation of the particular device, one of skill in the art should have little trouble combining the features of certain of two such devices. Therefore, it should be understood that many of the elements are interchangeable, and the invention covers all permutations thereof.

Other objects, features, and advantages of the present invention will become apparent from a consideration of the following detailed description.

### BRIEF DESCRIPTION OF THE DRAWINGS

Various examples are depicted in the accompanying drawings for illustrative purposes, and should in no way be interpreted as limiting the scope of the inventions. In addition, various features of different disclosed examples can be combined to form additional examples, which are part of this disclosure. Throughout the drawings, reference numbers may be reused to indicate correspondence between reference elements.

FIG. 1 illustrates an example representation of a heart and associated artery having various features relevant to certain examples of the present inventive disclosure.

FIGS. 2A and 2B depict a blood vessel having a blood vessel wall that is elastic.

FIGS. 3A and 3B depict a blood vessel having a blood vessel wall that is inelastic and/or has lost elasticity.

FIGS. 4A and 4B depict a compliant stent implanted within a blood vessel in accordance with one or more examples.

FIGS. 5A-5D depict views of a stent according to some examples.

FIG. 6 provides an overhead view of an example stent in a default and/or resting form and in an expanded and/or in an expanded form, in accordance with one or more examples.

FIGS. 7A and 7B depict a blood vessel with a stent portion having a lumen through which blood can freely flow according to one or more examples.

FIG. 8 depicts an example stent situated within a blood vessel in accordance with one or more examples.

FIG. 9A depicts an example of a system with a delivery catheter for deploying a stent.

FIGS. 9B, 9C, and 9D depict side views of deployment of the stent within a blood vessel using the delivery catheter.

FIG. 10 provides an overhead view of an example stent in a default and/or resting form and in an expanded and/or in an expanded form, in accordance with one or more examples.

FIG. 11 provides an overhead view of an example stent comprising angled support arms in a default and/or resting form and in an expanded and/or in an expanded form, in accordance with one or more examples.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

In humans and other vertebrate animals, blood circulation throughout the body is facilitated by a blood circulatory system comprising various arteries, capillaries, veins, and coronary vessels, which work together with the heart to supply blood to the various regions of the body. The heart generally comprises a muscular organ having four pumping chambers, wherein the flow thereof is at least partially controlled by various heart valves, namely, the aortic, mitral (or bicuspid), tricuspid, and pulmonary valves. The valves may be configured to open and close in response to a pressure gradient present during various stages of the cardiac cycle (e.g., relaxation and contraction) to at least partially control the flow of blood to a respective region of the heart and/or to blood vessels (e.g., pulmonary, aorta, etc.). The valves may permit fluid flow between the heart and the various arteries of the cardiovascular system.

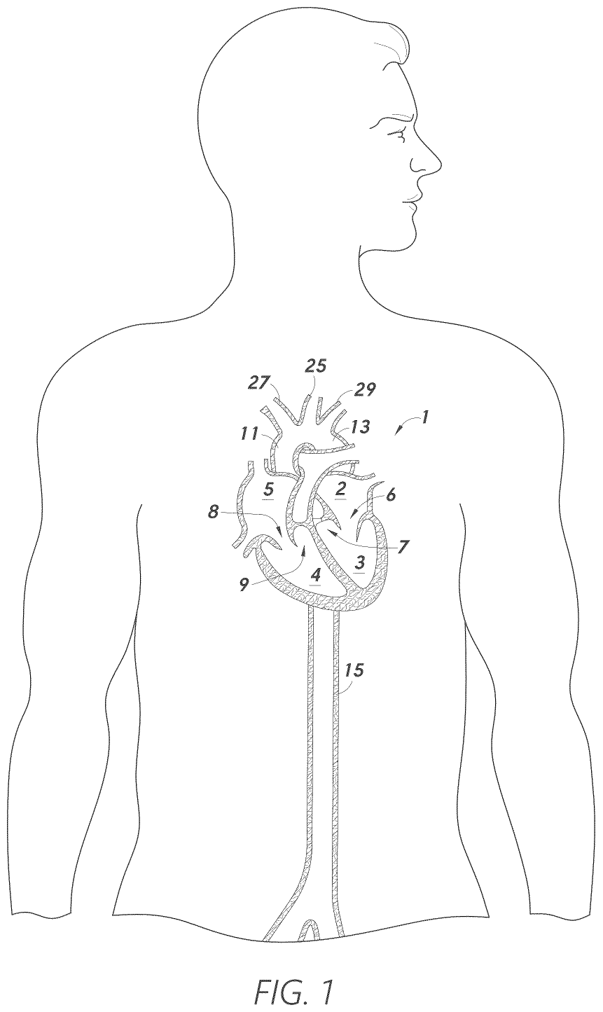

FIG. 1 illustrates an example representation of a heart 1 and associated artery 15 having various features relevant to certain examples of the present inventive disclosure. The heart 1 includes four chambers, namely the left atrium 2, the left ventricle 3, the right ventricle 4, and the right atrium 5. The heart 1 further includes four valves for aiding the circulation of blood therein, including the tricuspid valve 8, which separates the right atrium 5 from the right ventricle 4. The tricuspid valve 8 may generally have three cusps or leaflets and may generally close during ventricular contraction (i.e., systole) and open during ventricular expansion (i.e., diastole). The valves of the heart 1 further include the pulmonary valve 9, which separates the right ventricle 4 from the pulmonary artery, and may be configured to open during systole so that blood may be pumped toward the lungs, and close during diastole to prevent blood from leaking back into the heart from the pulmonary artery. The pulmonary valve 9 generally has three cusps/leaflets (not shown). The heart 1 further includes the mitral valve 6, which generally has two cusps/leaflets (not shown) and separates the left atrium 2 from the left ventricle 3. The mitral valve 6 may generally be configured to open during diastole so that blood in the left atrium 2 can flow into the left ventricle 3, and advantageously close during diastole to prevent blood from leaking back into the left atrium 2. The aortic valve 7 separates the left ventricle 3 from the aorta 11. The aortic valve 7 is configured to open during systole to allow blood leaving the left ventricle 3 to enter the aorta 11, and close during diastole to prevent blood from leaking back into the left ventricle 3.

The aorta is coupled to the heart via the aortic valve 7, wherein the ascending aorta 11 arises from the heart 1 and gives rise to the innominate artery 27, the left common carotid artery 25, and the left subclavian artery 29 before continuing as the descending thoracic aorta 13 and further the abdominal aorta 15.

Arteries, such as the aorta 15, may utilize arterial compliance to store and release energy through the stretching of blood vessel walls. As described herein, arterial “compliance” may refer to the ability of an arterial blood vessel to distend and increase in volume with increasing transmural pressure, or the tendency of an artery, or portion thereof, to resist recoil toward its original dimensions on application of a distending or compressing force.

In a normal compliant blood vessel, volume expansion and contraction occur by the stretching and retraction of the blood vessel walls responsive to heart beats. Aging, hypertension and other factors may result in decreased elasticity in blood vessel walls in the vascular system, leading to decreased vascular compliance. Such decreased vascular compliance (also called arterial stiffness or vascular stiffness) may result in minimal volume change during the diastolic to systolic pressure change. Depending on where the decreased vascular compliance occurs, the patient's health can be impaired. Loss of compliance in the aorta 15, for example, can lead to increased pulse pressure (e.g., increased systolic pressure and/or decreased diastolic pressure) and can result in increased left ventricle workload (and/or decreased cardiac efficiency). The loss in aortic compliance can also have a detrimental effect on coronary perfusion. Treatment to improve vascular compliance restoration can include the addition of a compliant chamber that attaches to the vascular system, with the compliant chamber providing added compliance. Such chambers may introduce challenges, such as thrombosis and fatigue issues.

The present invention provides systems, devices, and methods for restoring compliance to non-compliant vascular structures by causing temporary changes in the cross-sectional geometry of a blood vessel without changing or with minimal change to a peripheral wall length of the blood vessel during initial implant. More specifically, the relaxed/diastolic cross-sectional shape of the blood vessel may be altered into a non-circular shape and/or, when subjected to increased pressure (e.g., during systole), the blood vessel may assume a circular (or more circular) shape. Because a highest area-to-periphery ratio may be achieved with a circular shape, a device which provides for transition between a non-circular shape and a circular (or more circular) shape may allow for cross-sectional area expansion. Accordingly, some examples herein may allow for expansion/contraction of the blood vessel cross-sectional area between diastole/systole without the need for high elasticity of the blood vessel walls.

Some example systems may comprise an elastic spring-like device that can distort (e.g., via pushing, pulling, etc.) the cross-sectional shape of the blood vessel to a cross-sectional shape that may have a smaller cross-sectional area at diastole (i.e., during the lower applied pressure). At systole (i.e., during the higher applied pressure), the elastic device can expand or otherwise flex to increase the cross-sectional area of the blood vessel (e.g., via change in blood vessel and/or device geometry but with little or no change in blood vessel and/or device perimeter) to provide the desired added compliance.

In some examples, a stent and/or similar device may provide an added compliance (i.e., added change in volume over a constant change in pressure) to any blood vessel in or on which it is placed. The term “stent” is used herein in accordance with its broad and ordinary meaning and may refer to any device configured to be implanted in a blood vessel for improving compliance of the blood vessel. Such added compliance can benefit any part of a pulsatile flow system that may suffer from increased systolic pressures (e.g., hypertension). Some example devices may be configured to shift systolic flow to diastolic flow with a constant cardiac output (e.g., yielding increased cardiac efficiency), which may be highly beneficial in the aorta and/or for coronary perfusion. Examples of blood vessels that can benefit from added compliance can include the aorta, pulmonary arteries, and/or the superior/inferior vena cava.

In some examples, a flexible stent and/or other implant may be used to reshape a non-compliant/non-elastic blood vessel into a non-circular shape when in the diastolic/relaxed/biased condition. The non-circular shape may include an oval, triangle, peanut, figure-8, etc. The stent and/or other implant may be configured to be biased toward the non-circular shape and/or may be capable of deforming to a circular/more circular shape (and thus a larger cross-sectional area) in response to pressure. For example, blood flow through a non-compliant/non-elastic blood vessel may be capable of causing deformation of a stent and/or similar device described herein. The perimeter of the non-circular shape may be identical or similar to the more circular shape. In this way, the stent and blood vessel may maintain the same perimeter and/or have minimal change in perimeter while changing shape in response to blood flow changes. In some examples, the non-circular shape may have a smaller cross-sectional area than the more circular shape. When the heart beats, the blood vessel may be deformed into a more circular shape, after which the stent/implant may be configured to press and/or pull the blood vessel back toward the more non-circular shape. In this manner, the stent/implant may be configured to restore some compliance to the otherwise non-compliant blood vessel.

In some examples, a stent may be deployed within a blood vessel. However, one or more stents may additionally or alternatively be configured to be positioned around an outer surface of the blood vessel. A stent may comprise one or more hooks and/or other attachment mechanisms adapted to help secure the stent to the tissue of the blood vessel wall.

A stent may comprise a stent wall defining an elongated tubular member having a first end with a first opening. The tubular member may further comprise a second end with a second opening, a lumen extending between the first opening and the second opening, and/or a stent length extending between the first end and the second end. The stent wall may comprise an open cell wall and/or may be adapted to be secured to a blood vessel wall of a blood vessel, such as via outward-directed hooks and/or endothelialization. The stent wall and/or a lumen at least partially surrounded by the stent wall may be configured to form a cross-sectional shape, a cross-sectional area, a major dimension, and/or a minor dimension. The stent may be elastically deformable between a first configuration and a second configuration, with the stent biased toward the first configuration. The first configuration may define various characteristics of the stent, for example the major dimension may be a first major dimension, the minor dimension may be a first minor dimension, the cross-sectional area may be a first cross-sectional area, and the cross-sectional shape may be a first cross-sectional shape. The second configuration may define various characteristics of the stent, for example, the major dimension may be a second major dimension, the minor dimension may be a second minor dimension, the cross-sectional area may be a second cross-sectional area, and the cross-sectional shape may be a second cross-sectional shape. The first minor dimension may be less than the second minor dimension, the first major dimension may be greater than the second major dimension, and/or the first cross-sectional area may be smaller than the second cross-sectional area.

In some examples, one or more stents may be at least partially composed of a shape-memory material, such as Nitinol. Stents may be configured to be biased toward a first cross-sectional shape. The first cross-sectional shape may be any shape, including an oval, triangle, peanut, figure-8, and/or kidney shape.

A stent may be configured to be percutaneously delivered to a blood vessel in a compressed configuration. Once within the blood vessel, the stent and/or stent wall of the stent may be configured to be radially expanded into direct surface contact with the blood vessel wall (e.g., the aortic wall of an aorta). In some examples, the stent may be configured to be expanded such that the perimeter of a lumen of the stent may approximate and/or exceed a perimeter of the blood vessel at least prior to expansion of the stent. In some cases, a stent configured to expand to an at least slightly greater perimeter than the native blood vessel may provide improved traction and/or resistance to migration within the blood vessel. Moreover, the stent having a perimeter approximate to and/or greater than the blood vessel may increase and/or ensure positive engagement with the blood vessel and/or to maximize a compliance effect. The stent wall and/or a portion of the stent wall may be configured to be endothelialized into the blood vessel wall. In some examples, the blood vessel may be an aorta, and/or the second cross-sectional area of the lumen may approximate a cross-sectional area of the aortic section in which the stent is deployed.

A stent may be adapted to be physically held in a particular configuration after radial expansion of the stent and/or stent wall within the blood vessel and into direct contact with the blood vessel wall, which can provide time for the stent to be secured to the blood vessel wall, e.g., via endothelialization. For example, a stent may comprise and/or may be configured to attach to a tension line which may be configured to physically hold the stent in a specific desired configuration, such as where the tension line is adapted to restrain the stent major dimension or minor dimension to a desired size (e.g., holding the stent in a more oval shape or in a more circular shape). The tension line may be configured to be dissolvable within blood of a patient. The tension line may be configured to be percutaneously removed from the stent by a user such as an interventional cardiologist.

Some systems described herein for providing compliance to a native blood vessel may include a catheter and/or an implant such as a stent. The catheter may comprise a catheter distal portion, a catheter proximal portion, and/or an elongate catheter body extending from the catheter distal portion to the catheter proximal portion. In some examples, the catheter may be adapted for the catheter distal portion to be percutaneously advanced within a patient's vasculature to a blood vessel. The catheter distal portion may comprise an expandable balloon adapted to radially expand the stent into contact with the blood vessel wall. The catheter distal portion may comprise a retractable sheath adapted to prevent radial expansion of the stent. For example, the catheter distal portion may be configured to prevent the stent from expanding from a first (e.g., compressed) configuration to a second (e.g., expanded) configuration.

Some example devices may include hybrid/composite structures, such as devices having stents adapted to secure the device within the patient's vasculature, combined with self-expanding/biased stents for deforming between smaller and larger cross-sectional areas responsive to blood pressure as the heart beats. For example, a device may comprise a distal stent which, in an expanded configuration, may comprise a distal stent lumen and distal stent cross-sectional area. The distal stent may comprise a distal stent wall with an open cell configuration adapted to directly engage a blood vessel wall of the blood vessel and/or to permit blood to flow from the distal stent lumen to the blood vessel wall. The device may also comprise a proximal stent of similar configuration to the distal stent, such as having an expanded configuration with a proximal stent lumen and a proximal stent cross-sectional area, and/or comprising a proximal stent wall having an open cell configuration adapted to directly engage the blood vessel wall and to permit blood to flow from the proximal stent lumen to the blood vessel wall. The device may further include a middle stent configured to be positioned between the distal stent and the proximal stent, the middle stent formed from a memory material and forming a middle stent lumen. The middle stent may be configured to be elastically deformable between a first configuration and a second configuration, and/or may be configured to be biased toward the first configuration, wherein in the first configuration the middle stent and/or middle stent lumen may comprise a first cross-sectional shape, a first cross-sectional area, a first major dimension, and a first minor dimension. The first minor dimension may be less than the first major dimension and/or the first cross-sectional area may be less than the distal stent cross-sectional area and/or less than the proximal stent cross-sectional area. The device may comprise a lining extending between the distal stent and the proximal stent and along a middle stent wall of the middle stent portion. The lining may be adapted to prevent the flow of blood therethrough.

In the second configuration, the middle stent and/or middle stent lumen may have a second cross-sectional shape, a second cross-sectional area, a second major dimension, and/or a second minor dimension, with the first minor dimension being less than the second minor dimension. The first major dimension may be less than the second major dimension. The distal and proximal stents may be adapted to be radially expanded into contact with a blood vessel wall such as an aortic wall of an aorta. The second cross-sectional area of the lumen may approximate a cross-sectional area of the aorta.

The distal and proximal stents may be at least partially composed of a plastically deformable material, such as stainless steel or a cobalt alloy, yielding a more circular cross-section. The middle stent may be at least partially composed of a shape-memory material (e.g., Nitinol). The first cross-sectional shape of the middle stent may have any shape, for example an oval, triangle, kidney, peanut, and/or figure-8 shape.

Some examples may relate to methods for restoring compliance to a blood vessel. A method may include providing a system comprising a delivery catheter and/or a stent, with the delivery catheter having a catheter distal portion, a catheter proximal portion, and a catheter elongated body. The delivery catheter may be adapted to be advanced into a patient's vasculature to position the catheter distal portion within a desired blood vessel. The stent may be any implant as disclosed herein. The method may involve advancing the catheter distal portion through a patient's vasculature to the desired blood vessel, positioning the catheter distal portion at a desired treatment site in the desired blood vessel, radially expanding the stent into contact with the blood vessel wall at the desired treatment site, and/or removing the delivery catheter from the patient's vasculature. The catheter distal portion may comprise an expandable balloon, wherein radially expanding the stent may involve expanding the balloon. The stent may be configured to be positioned on the expandable balloon when the catheter distal portion is advanced through the patient's vasculature. The catheter distal portion may comprise a sheath configured to slide at least partially over the stent, such that when the catheter distal portion is advanced through the patient's vasculature, the sheath may be configured to be positioned at least partially over the stent.

After radially expanding the stent into contact with the blood vessel wall at the desired treatment site, the stent may be configured to be physically held by a restraint in a desired configuration, such as being held in the first configuration or in the second configuration. After removal of the delivery catheter from the patient (e.g., including hours or days later and/or after the stent has been endothelialized or otherwise secured to the blood vessel wall), the restraint can be released from the stent so that the stent may be no longer held in the first or second configuration and/or can adopt either configuration and/or deform between the configurations. In some examples, the restraint may comprise a tension line. Releasing the restraint may involve cutting and/or removing the tension line. The tension line may be absorbable, and releasing the stent from the restraint may be configured to occur responsive to exposure of the absorbable tension line to blood of the patient. The absorbable tension line may be adapted to be absorbed over sufficient time such that the stent may be endothelialized or otherwise secured to the blood vessel wall before the absorbable tension line is absorbed and the stent is released from the restraint.

A stent may comprise a tubular/cylindrical shape, a simple hoop, a C-shaped clip/clamp, and/or a spring-like mechanism. In some examples, a stent may be at least partially composed of a shape-memory material such as Nitinol, which may allow the stent to deform to cause the blood vessel to assume a circular shape when subjected to external forces (e.g., forces caused by blood flow) and then return to a non-circular shape to reduce the blood vessel cross-sectional area.

In some examples, a stent may be at last partially self-expanding, such as where formed from a memory material such as Nitinol. The stent may be configured to be delivered via catheter to a desired position in, around, or adjacent the blood vessel. The catheter may comprise an expandable balloon to aid in deployment of the stent into firm initial contact with the blood vessel, such as where the balloon expands the stent to a deployment size/diameter larger than its programmed/biased condition. The deployment size/diameter may even be larger than the expanded size/diameter that the stent may later achieve when expanded due to blood flow/heartbeat. This deployment size/diameter may be sufficient to embed or otherwise secure the stent to the blood vessel wall. In some examples, the stent can be at least partially open-celled, such as to avoid blocking branch vessels. Whereas in another example, the stent can be completely covered, such as to isolate the aorta or other blood vessels from fluid pressure (e.g., regions of aneurysms, or compromised vessel walls).

Stents may comprise barbs and/or other attachment mechanisms which can prevent migration and/or can help hold the stent securely to a blood vessel wall in order to cause the blood vessel cross-sectional shape to change responsive to the stent shape change. Such barbs and/or other attachment devices may be adapted to engage the wall of the blood vessel in which the stent is deployed.

In some examples, a stent may comprise one or more anchors extending from the main stent body (such as any of the main stent bodies disclosed herein). Each of the one or more anchors may be adapted to be deployed into engagement with tissue of a branch blood vessel or other vascular structure, such as where a branch blood vessel branches away from the main blood vessel. The branch blood vessel may have a diameter that may be smaller than the diameter of the main blood vessel in which the main stent body is deployed. One or more of the anchors may extend from the main stent body at a position between the first end and the second end of the main stent body, or from the first end or from the second end of the main stent body. One or more of the anchors may be adapted to be deployed into contact with walls of a renal artery, or into the walls of an iliac artery. One or more of the anchors may comprise a wireform (e.g., formed from a shape-memory material) and/or may be adapted to pass within the branch blood vessel and/or adapted to engage the wall tissue of the branch blood vessel such as an iliac or renal artery. One or more of the anchors may comprise an anchor stent body with an anchor stent wall defining an anchor stent lumen, and/or the anchor stent body may be adapted to be radially expanded into contact with a wall of a branch blood vessel such as an iliac or renal artery. The anchor stent body may be at least partially composed of a shape-memory material and/or may be biased toward a configuration wherein the anchor stent lumen comprises a cross-sectional shape which is non-circular (e.g., an oval, triangle, peanut, or kidney shape). In some examples, the anchor stent body may have an overall length in the range from 0.5 and 7 cm.

In some examples, a stent may comprise one or more support arms configured to at least partially resist deformation of the stent in certain blood pressure conditions. For example, during diastolic pressure, the support arms may be configured to hold the stent in a relaxed form (e.g., a generally oval cross-sectional shape). During systolic pressure, the support arms may be configured to yield and/or stretch to allow the stent to deform to a second configuration (e.g., a generally circular cross-sectional shape). The stent may comprise any number of support arms and/or the support arms may be situated near a central axis of the stent and/or distal from the central axis of the stent. In some examples, one or more support arms may be configured to extend laterally across a lumen formed by the stent.

In some examples, a system including one or more stents as described herein may be used for endovascular repair (e.g., Abdominal Aortic Aneurysm (AAA) endovascular repair). For example, a catheter may be inserted into a blood vessel of a patient to deliver a stent at or near an aneurysm. The stent may be configured to act as a graft and/or may be configured to expand and/or to deform from a less circular shape to a more circular shape within the vessel to form a more stable channel for blood flow.

The systems, devices, and/or methods described herein can be utilized in various catheter-based procedures, including minimally invasive procedures and percutaneous procedures. In some examples the methods/systems/devices may involve trans-aortic deliveries through a small chest (or abdominal) incision. In other examples, the methods/systems/devices can be used in minimally invasive surgical procedures. In yet other examples, the methods/systems/devices can be used in percutaneous procedures, such as via a catheter or catheters into the patient's arterial system (e.g., through the femoral or brachial arteries).

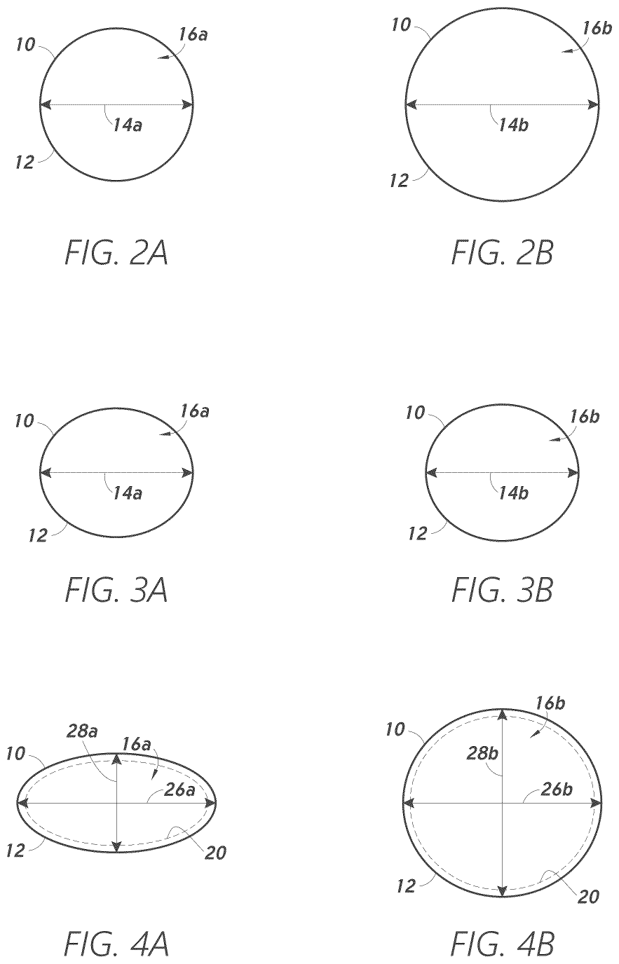

FIGS. 2A and 2B depict a blood vessel 10 having a blood vessel wall 12 that is elastic. The blood vessel 10 in diastolic pressure (FIG. 2A) may have a relatively small diameter 14a and cross-sectional area 16a, but in systolic pressure (FIG. 2B), the blood vessel wall 12 stretches so that the blood vessel 10 assumes a larger diameter 14b and cross-sectional area 16b.

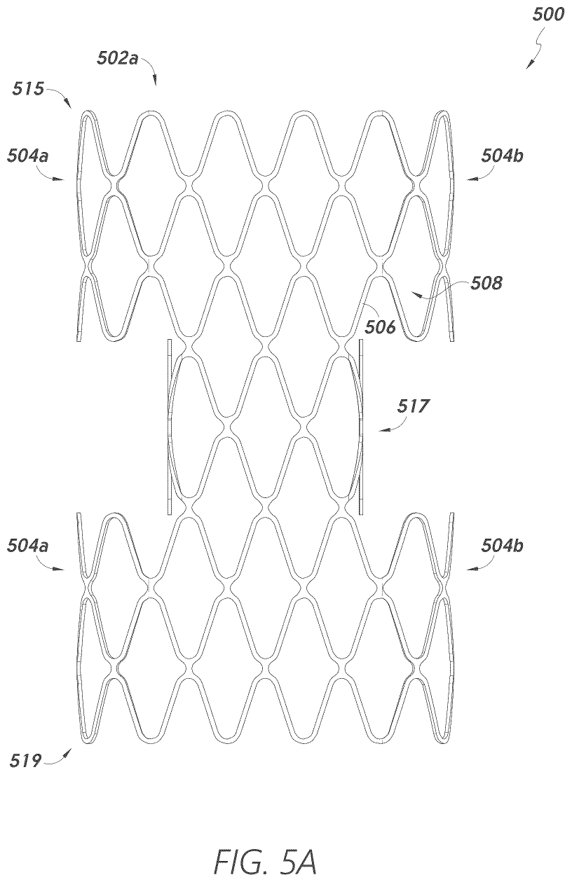

FIGS. 3A and 3B depict a blood vessel 10 having a blood vessel wall 12 that is inelastic and/or has lost elasticity. The diastolic diameter 14a and/or diastolic cross-sectional area 16a (FIG. 3A) may be only slightly smaller than the systolic diameter 14b and systolic cross-sectional area 16b (FIG. 3B).

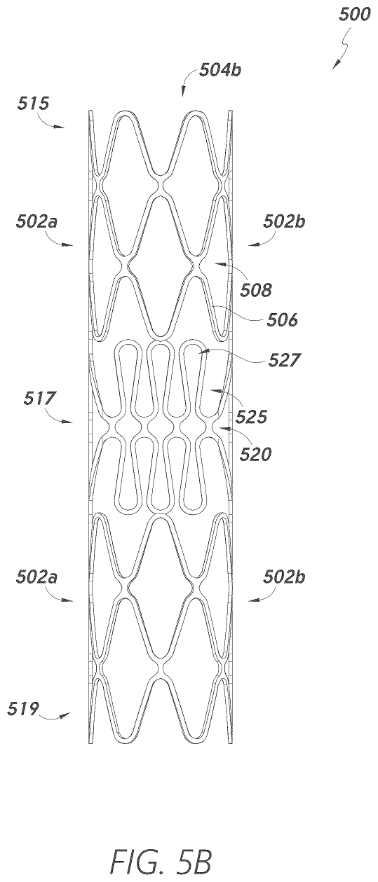

FIGS. 4A and 4B depict a compliant stent 700 implanted within a blood vessel 10. The stent 700 may be configured to restore compliance to the blood vessel 10. The stent 700 may comprise a stent wall configured to engage the blood vessel wall 12. In response to diastolic pressure, the stent 700 may be configured to have an oval diastolic shape with a major axis 26a and minor axis 28a. The stent 700 may be configured to cause the blood vessel 10 to assume a corresponding diastolic oval shape and/or diastolic cross-sectional area 16a. In response to systolic pressure, the stent 700 may be configured to assume a more circular systolic shape, which may cause the blood vessel wall 12 to assume a correspondingly more circular systolic shape where the stent minor axis 28b approaches or equals the stent major axis 26b, and/or with an enlarged systolic cross-sectional area 16b.

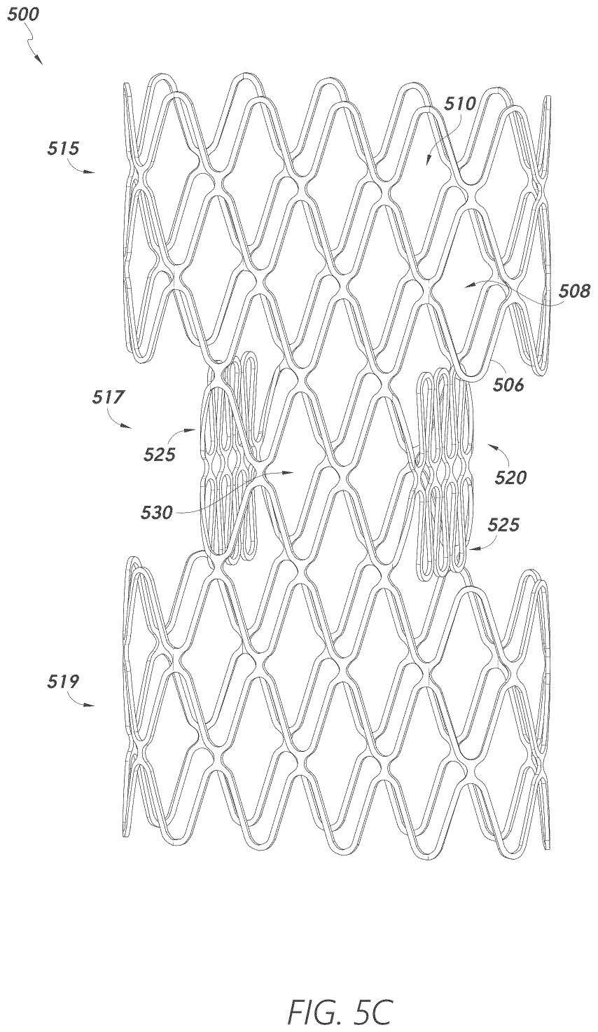

FIGS. 5A-5D depict views of a stent 500 according to some examples. FIG. 5A illustrates a first side view along a major axis of the stent 500, FIG. 5B illustrates a second side view along a minor axis of the stent 500, FIG. 5C provides a first perspective view of the stent 500, and FIG. 5D provides a second perspective view of the stent 500. As used herein, the terms “major” and “minor” are relative terms and/or the stent 500 may comprise a first major side 502a and/or a second major side 502b which may have greater lengths than a first minor side 504a and/or a second minor side 504b. In some examples, the first major side 502a may be situated generally across a lumen 510 of the stent from the second major side 502b. Similarly, the first minor side 504a may be situated generally across the lumen 510 of the stent 500 from the second minor side 504b. The first major side 502a and second major side 502b may extend along a first plane and/or the first minor side 504a and/or second minor side 504b may extend along a second plane.

The various components of the stent 500 described herein may be separate components and/or may comprise extensions of the same component. For example, the first major side 502a, the first minor side 504a, the second major side 502b, and/or the second minor side 504b may not be separate components and/or may refer to non-distinct portions of the stent 500. The stent 500 may not comprise distinct separation and/or transition points between components described herein. However, these components may be described in distinct terms for illustrative purposes herein.

The stent 500 may be configured to form a generally oval-shaped lumen 510 in a default and/or resting state of the stent 500. For example, the sides of the stent 500 may be at least partially curved, particularly at transition areas 512 between, for example, the first major side 502a and the first minor side 504a and/or second minor side 504b. In some examples, the first minor side 504a and/or the second minor side 504b may have more relatively more curvature than the first major side 502a and/or the second major side 502b at least at a default and/or resting form of the stent 500.

In some examples, the stent 500 may comprise a network of one or more struts 506 which may interconnect and/or form one or more cells 508 between the struts 506. One or more cells 508 formed by the struts 506 may have a generally diamond-shaped and/or rectangular form.

The stent 500 may comprise one or more support arms 520 configured to extend at least partially across the lumen 510 of the stent 500. The one or more support arms 520 may comprise a middle portion 517 of the stent 500. The middle portion 517 may be situated between a first end portion 515 and a second end portion 519 of the stent 500. The first end portion 515, middle portion 517, and/or second end portion 519 may each extend approximately a full length of the minor axis (e.g., from the first major side 502a to the second major side 502b). The first end portion 515 and/or the second end portion 519 may similarly extend approximately a full length of the major axis (e.g., from the first minor side 504a to the second minor side 504b). However, the middle portion 517 may not extend the full length of the major axis. In some examples, the middle portion 517 may extend approximately half of the length of the major axis. In other words, the stent 500 may have a generally constant width across the first end portion 515, middle portion 517, and/or second end portion 519 and/or may have a generally constant depth across the first end portion 515 and the second end portion 519. However, the stent 500 may have a smaller depth at the middle portion 517.

The middle portion 517 may form a generally oval-shaped inner lumen 530 that may have a smaller area than the lumen 510 formed by the first end portion 515 and/or the second end portion 519. In some examples, the inner lumen 530 may comprise a subsection of the lumen 510 of the stent 500.

The struts 506 may be configured to bend and/or navigate with respect to each other to provide flexibility to the stent 500. For example, the struts 506 may be configured to bend to allow the stent 500 to assume a generally compressed form within a catheter and/or other delivery device. Upon removal from the catheter and/or other delivery device, the stent 500 and/or struts 506 may be configured to relax and/or assume a default expanded and/or relaxed form. The stent 500 may be configured such that the first minor side 504a and/or second minor side 504b of the first end portion 515 and/or second end portion 519 may contact and/or exert force against a vessel wall when placed within a blood vessel.

The middle portion 517 may comprise one or more support arms 520 configured to extend at least partially across the lumen 510 of the stent 500. In some examples, the support arms 520 may comprise one or more tension springs 525 configured to naturally assume a coiled and/or compressed form and/or to expand and/or straighten in response to changes in blood pressure and/or blood pressure increasing above a threshold amount. The one or more tension springs 525 may have a generally wavy form in the resting/compressed/coiled form. Moreover, the one or more tensions springs 525 may have some rigidity and/or may be at least partially resistant to expansion and/or straightening. As a result, the one or more tensions springs 525 may be configured to deform, expand, and/or straighten only in response to increased pressure (e.g., in the range of 90-160 mmHg) and/or may return to the resting and/or coiled form following removal of such pressure.

The one or more tension springs 525 may be configured to form one or more loops 527 which may have droplet shapes to provide stability and/or a rigidity to the support arms 520. The tension springs 525 may be aligned in an accordion shape, as best shown in FIG. 5B.

As best shown in FIG. 5A, the stent 500 may have a variable width when viewed facing the first major side 502a and/or the second major side 502b. For example, the first end portion 515 and/or the second end portion 519 may have generally equal widths which may be greater than a width of the middle portion 517. The widths of each of the first end portion 515, middle portion 517, and/or second end portion 519 may be generally constant across each individual portion. In other words, the width of the stent 500 may not be tapered and/or may abruptly change from the first end portion 515 to the middle portion 517 and/or from the middle portion 517 to the second end portion 519.

As best shown in FIG. 5B, the stent 500 may have a generally constant width (i.e., depth) when viewed facing the first minor side 504a and/or the second minor side 504b. The support arms 520 may be configured to assume, in the relaxed and/or compressed form shown in FIGS. 5A-5D, a length approximately equal to a distance between the first major side 502a and the second major side 502b of the first end portion 515, middle portion 517, and/or second end portion 519.

The one or more support arms 520 may be configured to extend at least partially across a lumen formed by the first end portion 515 and/or the second end portion 519. For example, the first end portion 515 and/or the second end portion 519 may be approximately equal in size and/or may be aligned such that a lumen formed by the first end portion 515 extends linearly to a lumen formed by the second end portion 519. The one or more support arms 520 of the middle portion 517 may extend laterally across a space between the lumen formed by the first end portion 515 and the lumen formed by the second end portion 519. For example, blood flowing through the lumen of the first end portion 515 and/or the second end portion 519 may contact the one or more support arms 520. The one or more support arms 520 may comprise tension springs and/or at least partially coiled wire forms configured to increase in length in response to forces (e.g., blood pressure changes).

The first end portion 515, second end portion 519, and/or middle portion 517 may be configured to contact one or more blood vessel walls. For example, the first minor side 504a and/or second minor side 504b of the stent 500 and/or of the first end portion 515, middle portion 517, and/or the second end portion 519 may be configured to contact one or more blood vessel walls in the relaxed and/or unexpanded form shown in FIGS. 5A-5D. In response to blood flow and/or pressure changes, the one or more support arms 520 may be configured to expand and/or the first end portion 515 and/or second end portion 519 may be configured to bend and/or deform to a more circular form. As a result, the first major side 502a and/or second major side 502b of the stent 500 and/or of the first end portion 515 and/or second end portion 519 may be configured to contact one or more blood vessel walls and/or to exert a pressing force on the blood vessel walls.

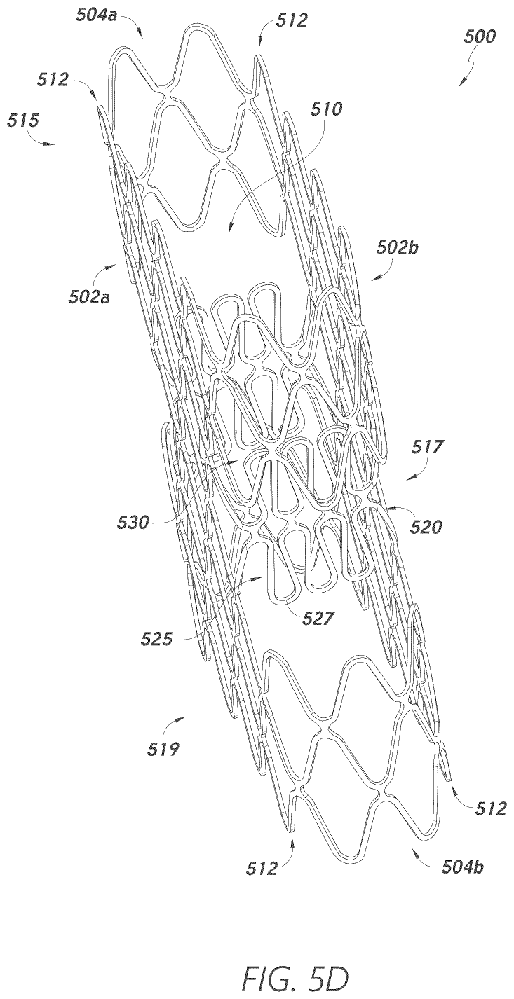

The stent 500 and/or the first end portion 515, middle portion 517, and/or second end portion 519 may comprise a network of elastically deformable struts 506 configured to bend and/or deform in response to blood flow and/or pressure changes. For example, the struts 506 may be configured to radially expand and/or the first end portion 515, middle portion 517, and/or second end portion 519 may be configured to radially expand from a first configuration (e.g., the resting and/or unexpanded configuration shown in FIGS. 5A-5D) to a second configuration (e.g., a more circular form; see FIG. 6). One or more portions of the stent 500 not in contact with one or more blood vessel walls in the first configuration may be moved into direct contact with one or more blood vessel walls in the second configuration. A perimeter of the stent 500 may remain unchanged between the first configuration and the second configuration. A cross-sectional area of the stent 500 may increase when moving from the first configuration to the second configuration.

A distance between the first major side 502a and the second major side 502b in the first configuration may define a first minor dimension of the stent 500. A distance between the first minor side 504a and the second minor side 504b in the first configuration may define a first major dimension. As the stent 500 moves from the first configuration to the second configuration, the stent 500 may assume a second minor dimension (e.g., distance between the first major side 502a and the second major side 502b) that is greater than the first minor dimension. Similarly, as the stent 500 moves from the first configuration to the second configuration, the stent 500 may assume a second major dimension (e.g., distance between the first minor side 504a and the second minor side 504b) that is smaller than the first major dimension.

The stent 500 may have, in the resting state shown in FIGS. 5A-5D, a generally oval-shaped cross-sectional shape. The perimeter of the stent 500 may exceed and/or approximate a perimeter and/or cross-sectional area of the aorta and/or other blood vessel. Accordingly, as the stent 500 deforms to a more circular cross-sectional shape in the second configuration, the sides of the stent 500 may increasingly press against the walls of the aorta and/or other blood vessel.

In the first configuration of the stent 500 shown in FIGS. 5A-5D, a length (e.g., distance between the first minor side 504a and the second minor side 504b) of the first end portion 515 and/or second end portion 519 (e.g., along the major axis) may be greater than a width (e.g., distance between the first major side 502a and the second major side 502b) of the first end portion 515 and/or the second end portion 519 (e.g., along the minor axis). The first end portion 515 and/or second end portion 519 may be configured to deform in response to blood flow and/or blood pressure changes such that the length of the first end portion 515 and/or second end portion 519 at least partially decreases and/or the width of the first end portion 515 and/or second end portion 519 at least partially increases.

The middle portion 517 may have a generally oval and/or rectangle shape. For example, the middle portion 517 may be generally flat along the first major side 502a and/or the second major side 502b. Moreover, the one or more support arms 520 of the middle portion 517 may extend generally linearly across the first major dimension of the stent 500. As a result, the middle portion 517 may comprise four generally straight sides. The corners and/or edges of the middle portion 517, first end portion 515, and/or second end portion 519 may be generally rounded.

A length of the middle portion 517 (e.g., a distance between the support arms 520 of the middle portion 517) in the first configuration may be less than the length of the first end portion 515 and/or of the second end portion 519. A width of the middle portion 517 (e.g., a distance between the first major side 502a and the second major side 502b) in the first configuration may be approximately equal to the width of the first end portion 515 and/or the second end portion 519. In the first configuration, the length of the middle portion 517 may be approximately half, seventy-five percent, and/or any other amount less than one hundred percent of the length of the first end portion 515 and/or the length of the second end portion 519. In the second configuration, the width of the middle portion 517 may increase beyond the width of the first end portion 515 and/or the second end portion 519 as the stent 500 assumes a more circular form.

The middle portion 517 may comprise a first side extending along the first major side 502a of the stent 500 and/or a second side extending along the second major side 502b of the stent 500. The first side and/or second side of the middle portion 517 may be coplanar with corresponding sides of the first end portion 515 and the second end portion 519.

The one or more support arms 520 may be generally straight. The first end portion 515 and/or second end portion 519 may comprise generally rounded ends and/or edges along the first minor side 504a and/or second minor side 504b and/or along a minor axis of the stent 500.

While the first end portion 515 and the second end portion 519 are shown having generally straight sides (e.g., along the first major side 502a, second major side 502b, first minor side 504a, and/or second minor side 504b), the first end portion 515 and/or second end portion 519 may have tapered and/or slanting sides. For example, the first end portion 515 and/or second end portion 519 may taper and/or reduce in width along the first minor side 504a and/or second minor side 504b such that a width of the first end portion 515 and/or second end portion 519 is minimal at or near the middle portion 517.

In some examples, at least a portion of the stent 500 and/or at least a portion of the struts 506 and/or cells 508 of the stent 500 may be at least partially covered by a covering. The covering may be configured to increase friction of the struts 506 and/or to close the cells 508 to reduce blood flow through the cells 508.

The one or more support arms 520 may be at least partially composed of Nitinol and/or other materials and/or alloys having shape memory features. For example, a Nitinol support arm 520 may be configured to increase in strain before reaching a plateau (e.g., approximately 8% strain) at which deformation of the support arm 520 may occur. In this way, the support arms 520 may be resistant to diastolic pressure and/or may be configured to deform and/or expand only in response to systolic and/or increased pressure and/or pressure at or above a given amount.

While the stent 500 is shown comprising two support arms 520, the stent 500 may comprise any number of support arms 520. For example, the stent 500 may comprise four support arms 520.

FIG. 6 provides an overhead view of an example stent 600 in a default and/or resting form 605 (illustrated in FIG. 6 using solid lines) and in an expanded and/or in an expanded form 607 (illustrated in FIG. 6 using dashed lines), in accordance with one or more examples. The stent 600 may be configured to expand from the resting form 605 to the expanded form 607 in response to changes in pressure within and/or around a vessel and/or other anatomy in which the stent 600 is situated.

The stent 600 may comprise one or more support arms 620 configured to extend across a lumen 610 formed by the stent 600 and/or formed by a first end portion and second end portion of the stent 600. A middle portion of the stent 600 may form an inner lumen 630 (e.g., between the support arms 620 of the middle portion). In some examples, the stent 600 may have, in the resting form 605, a generally oval-and/or pill-shaped form in which one or more end portions of the stent 600 form rounded edges 604 (e.g., the minor sides 504a, 504b of FIG. 5) and/or in which the one or more end portions and/or a middle portion form generally straight sides 602 (e.g., the major sides 502a, 502b of FIG. 5). The stent 600 may comprise a first major side 602a, a second major side 602b, a first minor side 604a, and/or a second minor side 604b.

When the stent 600 experiences pressure changes, the stent 600 may be configured to assume a more curved and/or generally circular-shaped expanded form 607. As shown in FIG. 6, the expanded form 607 may cause the support arms 620 to be extended to extend across a greater width of the stent 600. The extension of the support arms 620 may be enabled by bending and/or navigation of the struts 606 forming the stent 600 and/or by a length of the middle portion of the stent 600 being reduced.

In the relaxed form 605, the stent 600 may have a relatively small cross-sectional area. The stent 600 may be configured to expand to the expanded form 607 in response to increased aortic blood pressure (e.g., during systole). The stent 600 may as a result assume a more circular cross-section, which can increase the cross-sectional area of the stent 600 (e.g., by bending and/or movement of the struts 606 of the stent 600) while generally maintaining the circumference of the stent 600. The increase in cross-sectional area can facilitate absorption of the pulsatile shock of systole. After systole, the stent 600 and/or blood vessel can return to the relaxed form 605 and/or to a generally oval shape. The return to the relaxed form 605 can force blood through the aorta. As a result, blood pressure pulses through the aorta can be smoothed to imitate performance of a more compliant aorta.

The stent 600 portion may, in the resting and/or default form 605, have a generally non-circular (e.g., oval) shape with a major axis 26a and minor axis 28a. As blood pressure increases in the blood vessel 10, the stent 600 may be configured to allow blood to pass through the lumen 610 and/or press against the blood vessel wall 12. During periods of increased pressure, the stent 600 may be configured to compress at least partially along the major axis 26a of the stent 600, causing the stent 600 to assume a less oval/more circular shape of the expanded form 607, where the major axis 26b may be shorter than it was prior to the increase of blood pressure and/or the blood vessel 10 may have a larger cross-sectional area than in the more oval shape of FIG. 6.

While the struts 606 forming the stent 600 may be at least partially flexible to enable changing cross-sectional area of the stent 600, the struts 606 may be at least partially resistant to changes in shape. For example, if the stent 600 is too responsive (e.g., too flexible), the stent 600 may not sufficiently resist blood pressure increases during systole and/or can change shape prematurely. The struts 606 may thus be configured to bend and/or change shape in response to pressure exceeding a threshold amount. In some examples, the struts 606 may be thickened, however thickening of the struts 606 may cause increased stress on the material of the struts 606 as the struts 606 bend.

The middle portion of the stent 600 may be configured to extend at least partially across the lumen 610 of the stent 600 to form supporting arms and/or chords for the stent 600. As a result, the middle portion can provide increased resistance to circularizing and/or can increase a force to pull the stent 600 back to the relaxed form 605 following systole.

At least part of the middle portion (e.g., the support arms) may be formed by tension springs and/or struts 606 having more curved shapes to provide increased spring behavior for the support arms extending across the lumen 610 of the stent 600. The tension springs of the support arms can be tuned (e.g., by their geometry and/or using thermal treatment of the struts 606) to allow the support arms to have spring behavior at least partially independently of the tubular geometry of the stent 600. In some examples, the support arms may and/or other struts 606 of the stent 600 may be at least partially composed of one or more shape memory alloys (e.g., Nitinol) to allow the support arms to have relatively high resistance until a threshold level of blood pressure and/or to allow for movement from the relaxed form 605 to the expanded form 607 when the threshold level of blood pressure is reached. For example, the stent 600 may be configured to move from the relaxed form 605 to the expanded form 607 at a given point during systole and/or not immediately at the beginning of systole. For example, the support arms may be tuned such that martensitic stress is reached at the threshold blood pressure.

The oval shape of the stent 600 at the relaxed form 605 may provide for a relatively high cross-sectional change along the minor axis of the stent 600 between the relaxed form 605 and the expanded form 607. Most of the volume change of the stent 600 may be applied above diastolic pressure and/or not immediately at the beginning of systole. In some examples, the middle portion of the stent may comprise one or more biphasic springs.

In some examples, the stent 600 may be formed from a laser-cut hypotube.

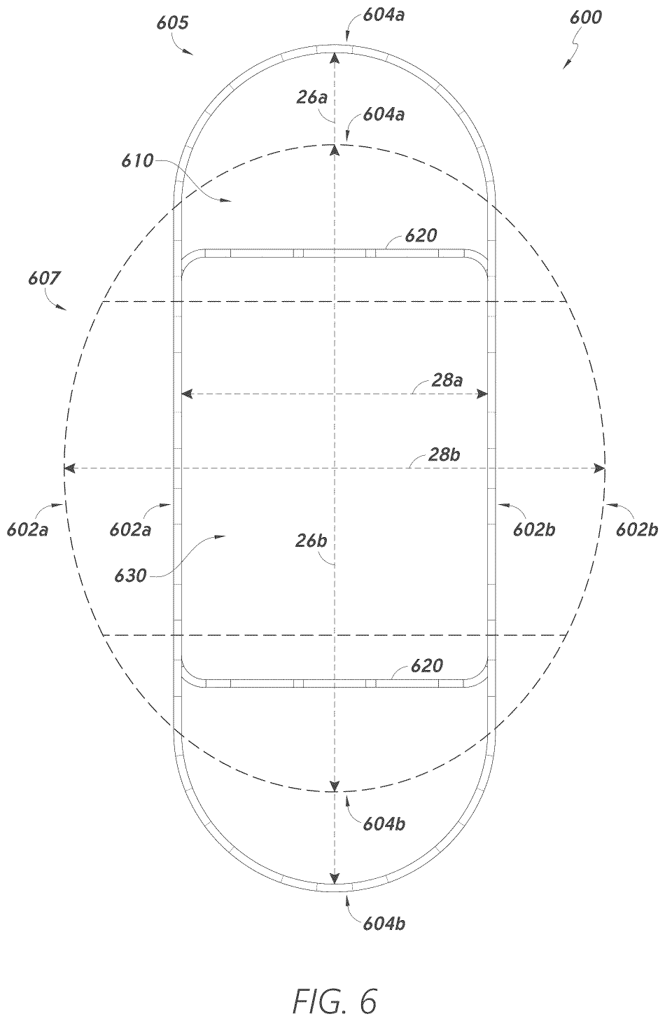

FIGS. 7A and 7B depict a blood vessel 10 with a stent 700 portion having a lumen 710 through which blood can freely flow. In FIG. 7, the stent 700 portion may have a generally non-circular (e.g., oval) shape with a major axis 26a and minor axis 28a. As blood pressure increases in the blood vessel 10, the stent 700 may be configured to allow blood to pass through the lumen 710 and/or press against the blood vessel wall 12. The blood pressure may accordingly create tension 48 in the blood vessel wall 12, particularly in the blood vessel wall portions running roughly parallel to the stent major axis 26a. The tension 48 may result in the blood vessel 10 compressing the stent 700 along the major axis 26a of the stent 700, causing the stent 700 to assume a less oval/more circular shape as depicted in FIG. 7B, where the major axis 26b may be shorter than it was prior to the increase of blood pressure (shown in FIG. 7) and/or the blood vessel 10 may have a larger cross-sectional area than in the more oval shape of FIG. 7. The stent 700 may comprise one or more support arms 720 extending laterally across a lumen of the stent 700.

For a closed/covered stent 700, blood may be prevented from flowing through the stent 700 to press outwardly against the blood vessel wall 12. Instead, pressure load may act directly onto the stent 700 via a lining material of the stent 700 (e.g., polymer film, bio-prosthetic material, cloth, etc.). For such closed/covered/lined stents 700, one or more materials of the stent 700 may create a sealing between the stent 700 and the blood vessel wall 12 and/or between the stent 700 and one or more adjacent stents which may be deployed in the same blood vessel 10. Such scaling can be achieved using deflections in the outer surface of the stent 700, such as in the form of raised and/or lowered features in the outer surface of the stent 700. Examples of such features can include raised and/or lowered features (e.g., raised bumps or edges or lowered troughs) at the distal and/or proximal ends of the stent 700 for improved sealing at the leading and/or trailing stent edges and/or against the native anatomy. Raised and/or lowered features such as bumps and/or ridges and/or troughs may be configured to be positioned at various positions along the length of the stent 700. Raised and/or lowered features may be formed in various ways, for example using compliant materials such as cloth, foam, elastomers, etc.

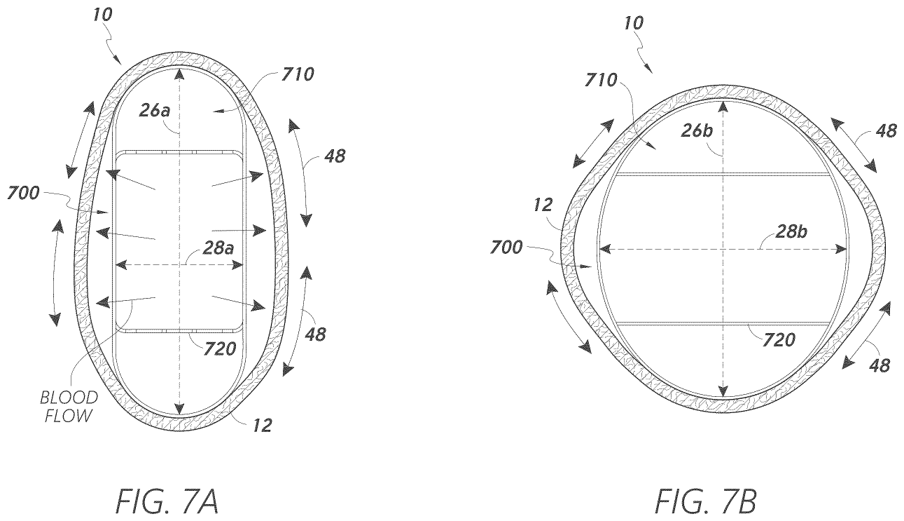

FIG. 8 depicts an example stent 800 situated within a blood vessel 10 in accordance with one or more examples. The stent may be open (i.e., may allow blood flow through the stent 800). In some examples, the stent 800 may be configured to at least partially contact and/or apply force to one or more blood vessel walls 12 of the blood vessel 10.

The stent 800 may comprise a first end portion 815, a middle portion 817, and/or a second end portion 819. In some examples, the first end portion 815 and/or second end portion 819 may be configured to engage and/or contact one or more walls 12 of the blood vessel 10. The middle portion 817 may have a width that is less than the width of the first end portion 815 and/or second end portion 819 and/or may not be configured to contact the walls 12 of the vessel 10. The middle portion 817 may comprise one or more expandable support arms 820 formed by tension springs and/or similar mechanisms.

While in some examples, a stent 800 may comprise a single length of linear tube, stents may comprise multiple lengths of linear tube and/or other forms. For example, a stent 800 may comprise Y-shaped structures wherein a stent with a bifurcated end may be provided to fit an anatomical bifurcation. In such an example, various portions of the Y-shape may be biased toward the same general shape and/or may be biased toward different shapes. For example, a main base/leg of the Y-shape structure may be biased toward a first shape (e.g., oval shape), a left upper arm portion of the Y-shape may be biased toward a second shape (e.g., kidney shape), and/or a right upper arm portion of the Y-shape may be biased toward a third shape (e.g., circular shape). Any combination of the shapes disclosed herein can be applied to the various portions of a device of the various examples described herein.

In some examples, the stent 800 may comprise one or more sensors 804, which can include pressure sensors and/or other sensors. Aging and/or other factors can result in decreased elasticity in the arterial system and/or can result in loss of vascular compliance (e.g., arterial stiffness and/or vascular stiffness). Loss of compliance in the aorta, for example, can lead to increased pulse pressure (e.g., systolic pressure) and/or can result in increased left ventricle workload. Loss in aortic compliance can also have a detrimental effect on coronary perfusion. The addition of one or more sensors 804 coupled to the stents 800 described herein can help quantify and/or monitor the effects of restored compliance and/or reduced stiffness. Sensors 804 can be incorporated into any of the stents described herein.

Loss of aortic compliance can increase central pulse pressure and/or systolic pressure and/or decrease diastolic pressure. One or more on-board pressure sensors 804 can provide accurate central blood pressure measurements (e.g., pressure in the aorta) and/or can monitor pressure benefits of restored compliance. A pressure sensor 804 can be positioned on an oval shaped stent 800 such that the sensor 804 may be at least partially exposed to aortic pressures prior to full deployment of the stent 800. For example, the sensor 804 can be positioned at a distal end of the stent 800 during transcatheter delivery of the stent 800. The sensor 804 can provide basal pressure data to measure resulting pressure changes due to the deployment of the stent 800 and/or restoration of aortic compliance.

One or more sensors 804 can be used to track movements of the stent 800 and/or can provide quantitative measurements of a change in volume from a systolic to a diastolic shape of the stent 800. The one or more sensors 804 can comprise proximity and/or position sensors producing volume change data that can be coupled with pressure change data yielding quantitative data on an amount of compliance restored.

The one or more sensors 804 can comprise temperature sensors configured to provide central temperature measurements that can be useful in a variety of medical applications. For example, temperature sensors can be used to estimate blood flow in conjunction with an induced current.

While the sensor 804 is shown coupled to the second end portion 819 in FIG. 8, one or more sensors 804 may be coupled to any portion of the stent 800, including the first end portion 815 and/or the middle portion 817. Sensors 804 may be coupled to the stent 800 using any suitable attachments devices and/or mechanisms. For example, as shown in FIG. 8, one or more coils 813 may be used to at least partially enclose the sensor 804 and/or one or more struts 809 of the stent 800 to secure the sensor 804 to the stent 800.

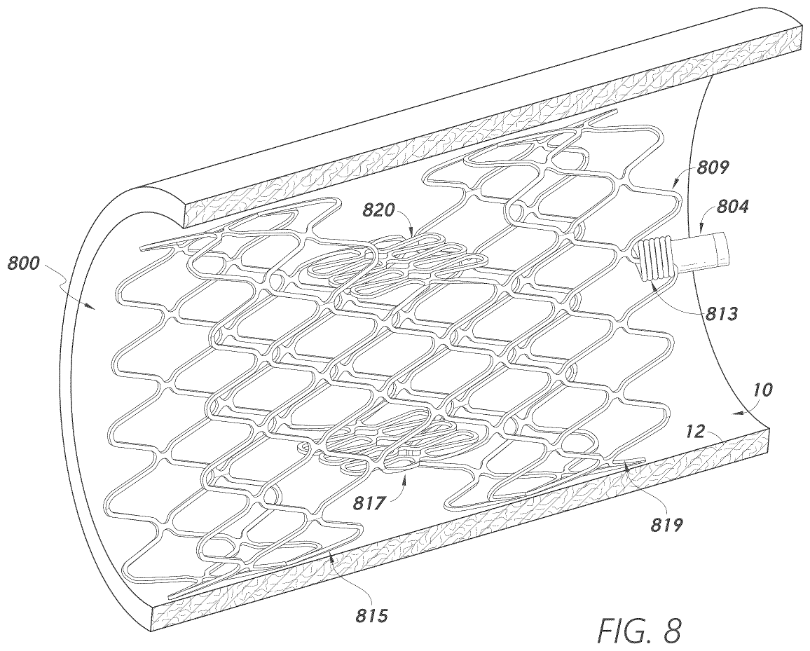

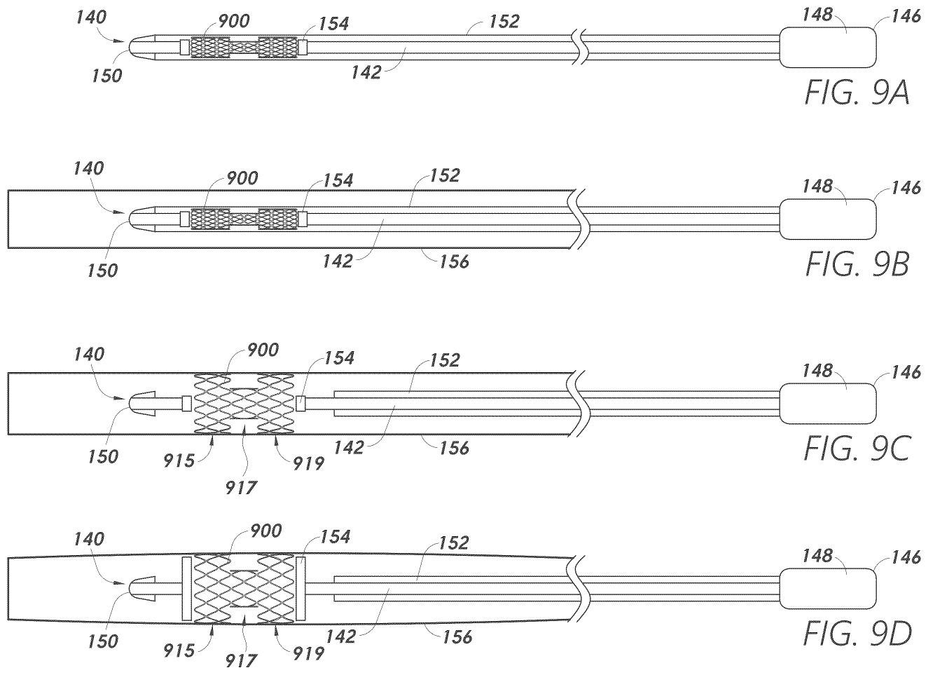

FIG. 9A depicts an example of a system 140 with a delivery catheter 142 for deploying a stent 900. The delivery catheter 142 may comprise a proximal end 146 with a handle 148 with controls thereon. The delivery catheter 142 may further comprise a distal end 150 adapted to be advanced into a blood vessel, such as via percutaneous methods. The system 140 may further comprise a retractable sheath 152 configured to cover and/or protect the stent 900 during delivery. In some examples, the sheath 152 may be configured to be retracted to permit the stent 900 to expand at a desired deployment position. The catheter 142 may comprise an expandable balloon 154 which can be selectively expanded in order to expand the stent 900 firmly into contact with a blood vessel wall. For a self-expanding stent, the balloon 154 can add further expansion to firmly engage the stent 900 against a blood vessel wall. In some examples, components of the system 140 may be configured to be loaded onto the delivery catheter 142 via various methods. For example, radial crimping, folding, and/or rolling may be used to load onto the catheter 142.

FIGS. 9B, 9C, and 9D depict side views of deployment of the stent 900 within a blood vessel 156 using the delivery catheter 142. The distal end 150 of the delivery catheter 142 may be advanced into the blood vessel 156 to a desired deployment location, as depicted in FIG. 9B. The sheath 152 may be retracted, at which point a self-expanding stent 900 may be configured to radially expand, as shown in FIG. 9C. The balloon 154 may be expanded to expand the stent 900 and/or to over-expand a self-expanding stent 900 to firmly press the stent 900 against the wall of the blood vessel 156, as depicted in FIG. 9D. After the stent 900 is properly deployed, the catheter 142 can be withdrawn from the patient. The sheath 152 may be advanced to a closed position (e.g., back over the balloon 154) prior to withdrawal of the catheter 142. With the stent 900 deployed in the blood vessel 156, the stent 900 may be configured to deform the blood vessel 156 as desired during diastole and/or systole to vary the blood vessel cross-sectional area responsive to heart beats of the patient in order to restore some blood vessel compliance. The stent 900 may comprise a first end portion 915, a middle portion 917, and/or a second end portion 919.

Various approaches for treatments, including advancing the catheter 142 into position via the sheath 152, are within the scope of this disclosure. In some examples, artery access may be obtained via an access sheath 152 dimensioned for use in some procedures. An incision may be created in a patient, leading to an internal blood vessel 156 (e.g., a femoral artery). The distal end of the access sheath 152 may be advanced through the incision and internal blood and into a desired position within the target blood vessel 156, with the catheter handle 148 positioned outside the patient adjacent the incision/access site. Echo and/or fluoroscopic and/or other visualization techniques may be used to confirm proper position of the stent 900. The treatment and/or implant deployment can occur, such as by deploying the stent 900 at the target location. Once the proper deployment is confirmed, the catheter 142 can be removed from the patient, and the incision(s) may be closed, for example via sutures.

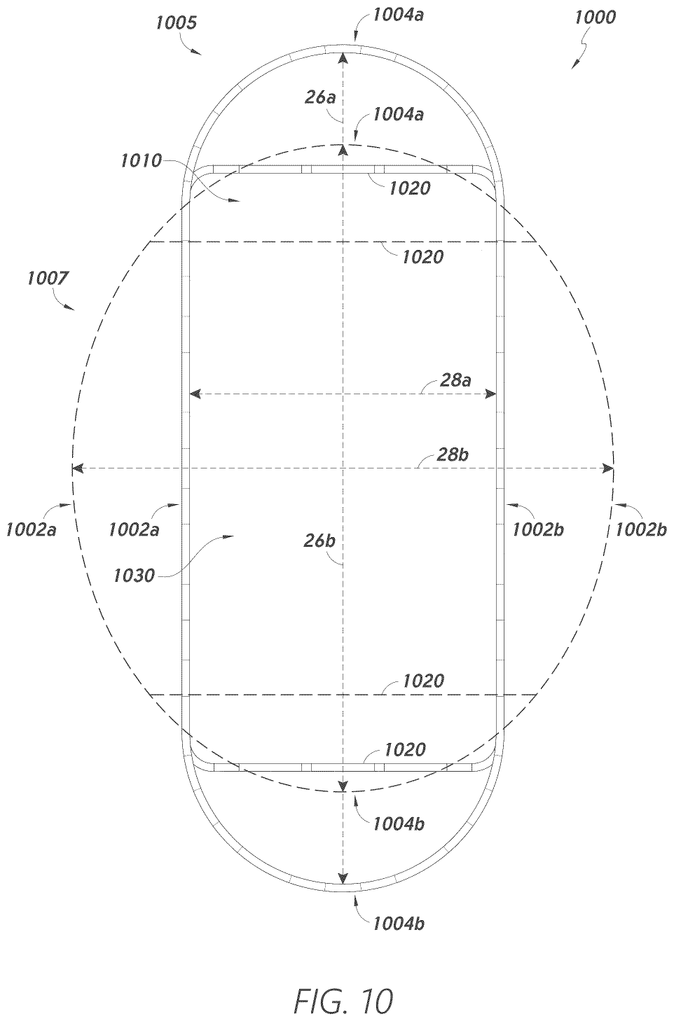

FIG. 10 provides an overhead view of an example stent 1000 in a default and/or resting form 1005 (illustrated in FIG. 10 using solid lines) and in an expanded and/or in an expanded form 1007 (illustrated in FIG. 10 using dashed lines), in accordance with one or more examples. The stent 1000 may be configured to expand from the resting form 1005 to the expanded form 1007 in response to changes in pressure within and/or around a vessel and/or other anatomy in which the stent 1000 is situated.

The stent 1000 may comprise one or more support arms 1020 configured to extend across a lumen 1010 formed by the stent 1000 and/or formed by a first end portion and/or second end portion of the stent 1000. A middle portion of the stent 1000 may form an inner lumen 1030 (e.g., between the support arms 1020 of the middle portion). In some examples, the stent 1000 may have, in the resting form 1005, a generally oval-and/or pill-shaped form in which one or more end portions of the stent 1000 form rounded edges 1004 (e.g., the minor sides 504a, 504b of FIG. 5) and/or in which the one or more end portions and/or a middle portion form generally straight sides 1002 (e.g., the major sides 502a, 502b of FIG. 5). The stent 1000 may comprise a first major side 1002a, a second major side 1002b, a first minor side 1004a, and/or a second minor side 1004b.

When the stent 1000 experiences pressure changes, the stent 1000 may be configured to assume a more curved and/or generally circular-shaped expanded form 1007. As shown in FIG. 10, the expanded form 1007 may cause the support arms 1020 to be extended to extend across a greater width of the stent 1000. The extension of the support arms 1020 may be enabled by bending and/or navigation of the struts forming the stent 1000 and/or by a length of the middle portion of the stent 1000 being reduced.

In the relaxed form 1005, the stent 1000 may have a relatively small cross-sectional area. The stent 1000 may be configured to expand to the expanded form 1007 in response to increased aortic blood pressure (e.g., during systole). The stent 1000 may as a result assume a more circular cross-section, which can increase the cross-sectional area of the stent 1000 (e.g., by bending and/or movement of the struts of the stent 1000) while generally maintaining the circumference of the stent 1000. The increase in cross-sectional area can facilitate absorption of the pulsatile shock of systole. After systole, the stent 1000 and/or blood vessel can return to the relaxed form 1005 and/or to a generally oval shape. The return to the relaxed form 1005 can force blood through the aorta. As a result, blood pressure pulses through the aorta can be smoothed to imitate performance of a more compliant aorta.

The stent 1000 portion may, in the resting and/or default form 1005, have a generally non-circular (e.g., oval) shape with a major axis 26a and minor axis 28a. As blood pressure increases in the blood vessel 10, the stent 1000 may be configured to allow blood to pass through the lumen 1010 and/or press against the blood vessel wall 12. During periods of increased pressure, the stent 1000 may be configured to compress at least partially along the major axis 26a of the stent 1000, causing the stent 1000 to assume a less oval/more circular shape of the expanded form 1007, where the major axis 26b may be shorter than it was prior to the increase of blood pressure and/or the blood vessel 10 may have a larger cross-sectional area than in the more oval shape of FIG. 10.

While the struts forming the stent 1000 may be at least partially flexible and/or deformable to enable changing cross-sectional area of the stent 1000, the struts may be at least partially resistant to changes in shape. For example, if the stent 1000 is too responsive (e.g., too flexible), the stent 1000 may not sufficiently resist blood pressure increases during systole and/or can change shape prematurely. The struts may thus be configured to bend and/or change shape in response to pressure exceeding a threshold amount. In some examples, the struts may be thickened, however thickening of the struts may cause increased stress on the material of the struts as the struts bend.

The middle portion of the stent 1000 may be configured to extend at least partially across the lumen 1010 of the stent 1000 to form supporting arms and/or chords for the stent 1000. As a result, the middle portion can provide increased resistance to circularizing and/or can increase a force to pull the stent 1000 back to the relaxed form 1005 following systole.

At least part of the middle portion (e.g., the support arms 1020) may be formed by tension springs and/or struts having more curved shapes to provide increased spring behavior for the support arms extending across the lumen 1010 of the stent 1000. The tension springs of the support arms can be tuned (e.g., by their geometry and/or using thermal treatment of the struts) to allow the support arms to have spring behavior at least partially independently of the tubular geometry of the stent 1000. In some examples, the support arms may and/or other struts of the stent 1000 may be at least partially composed of one or more shape memory alloys (e.g., Nitinol) to allow the support arms to have relatively high resistance until a threshold level of blood pressure and/or to allow for movement from the relaxed form 1005 to the expanded form 1007 when the threshold level of blood pressure is reached. For example, the stent 1000 may be configured to move from the relaxed form 1005 to the expanded form 1007 at a given point during systole and/or not immediately at the beginning of systole. For example, the support arms may be tuned such that martensitic stress is reached at the threshold blood pressure.

The oval shape of the stent 1000 at the relaxed form 1005 may provide for a relatively high cross-sectional change along the minor axis of the stent 1000 between the relaxed form 1005 and the expanded form 1007. Most of the volume change of the stent 1000 may be applied above diastolic pressure and/or not immediately at the beginning of systole. In some examples, the middle portion of the stent may comprise one or more biphasic springs.

The middle portion may have any suitable length and/or any suitable distance between support arms 1020 of the middle portion. For example, the one or more support arms 1020 may be situated near and/or adjacent to the minor sides 1004 of the stent 1000, as shown in FIG. 10. The length of the middle portion and/or any distance between support arms 1020 of the middle portion may be approximately half the length of the stent 1000 along the major axis 26a and/or may be greater than half the length of the stent 1000 (e.g., approximately seventy-five percent of the length of the stent 1000 and/or ¾ of the length of the stent 1000.

The support arms 1020 may be configured to support the stent 1000 without interfering with crimping of the stent 1000. In some examples, the one or more support arms 1020 may be vertically disposed between walls and/or sides of the first end portion and/or second end portion of the stent 1000. The one or more support arms 1020 may be at least partially offset from a central axis of the stent and/or may be near and/or adjacent to rounded minor sides 1004 of the stent 1000.

The stent 1000 may comprise any number of support arms 1020 and/or the support arms 1020 may have any suitable size. A spring constant and/or other geometrical features of a support arm 1020 may be selected to resist deformation of the stent 1000 when subjected to pressures that do not exceed a desired threshold value and/or may be configured to yield at elevated pressures (e.g., in the range of 90-160 mmHg).

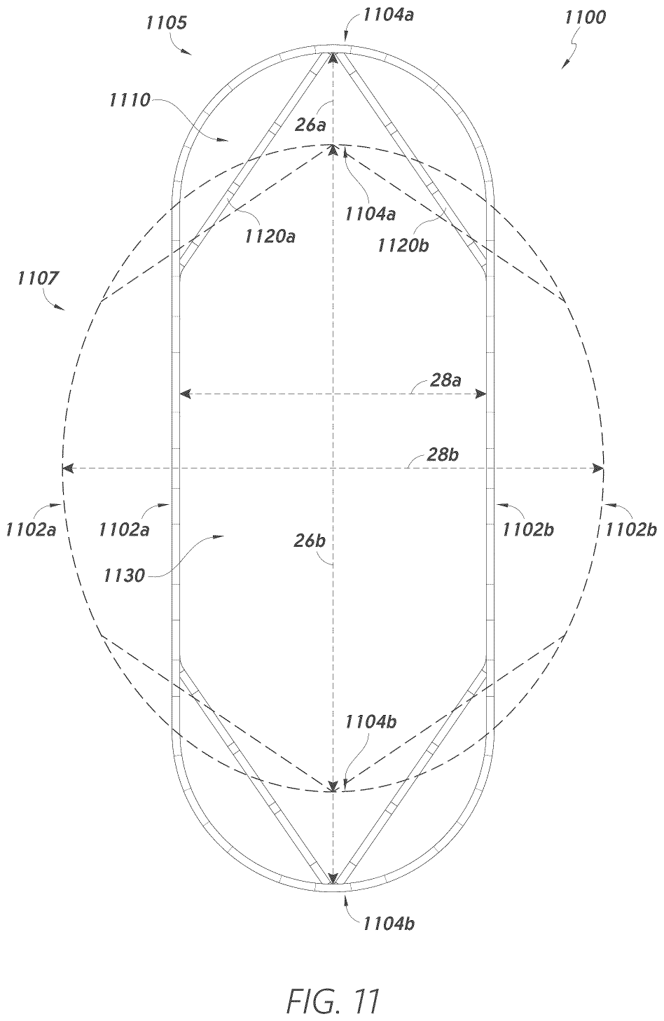

FIG. 11 provides an overhead view of an example stent 1100 in a default and/or resting form 1105 (illustrated in FIG. 11 using solid lines) and in an expanded and/or in an expanded form 1107 (illustrated in FIG. 11 using dashed lines), in accordance with one or more examples. The stent 1100 may be configured to expand from the resting form 1105 to the expanded form 1107 in response to changes in pressure within and/or around a vessel and/or other anatomy in which the stent 1100 is situated.

The stent 1100 may comprise one or more support arms 1120 configured to extend across a lumen 1110 formed by the stent 1100 and/or formed by a first end portion and/or second end portion of the stent 1100. A middle portion of the stent 1100 may form an inner lumen 1130 (e.g., between the support arms 1120 of the middle portion). In some examples, the stent 1100 may have, in the resting form 1105, a generally oval-and/or pill-shaped form in which one or more end portions of the stent 1100 form rounded edges 1104 (e.g., the minor sides 504a, 504b of FIG. 5) and/or in which the one or more end portions and/or a middle portion form generally straight sides 1102 (e.g., the major sides 502a, 502b of FIG. 5).

When the stent 1100 experiences pressure changes, the stent 1100 may be configured to assume a more curved and/or generally circular-shaped expanded form 1107. As shown in FIG. 11, the expanded form 1107 may cause the support arms 1120 to be extended to extend across a greater width of the stent 1100. The extension of the support arms 1120 may be enabled by bending and/or navigation of the struts forming the stent 1100 and/or by a length of the middle portion of the stent 1100 being reduced.

In the relaxed form 1105, the stent 1100 may have a relatively small cross-sectional area. The stent 1100 may be configured to expand to the expanded form 1107 in response to increased aortic blood pressure (e.g., during systole). The stent 1100 may as a result assume a more circular cross-section, which can increase the cross-sectional area of the stent 1100 (e.g., by bending and/or movement of the struts of the stent 1100) while generally maintaining the circumference of the stent 1100. The increase in cross-sectional area can facilitate absorption of the pulsatile shock of systole. After systole, the stent 1100 and/or blood vessel can return to the relaxed form 1105 and/or to a generally oval shape. The return to the relaxed form 1105 can force blood through the aorta. As a result, blood pressure pulses through the aorta can be smoothed to imitate performance of a more compliant aorta.

The stent 1100 portion may, in the resting and/or default form 1105, have a generally non-circular (e.g., oval) shape with a major axis 26a and minor axis 28a. As blood pressure increases in the blood vessel 11, the stent 700 may be configured to allow blood to pass through the lumen 710 and/or press against the blood vessel wall 12. During periods of increased pressure, the stent 700 may be configured to compress at least partially along the major axis 26a of the stent 1100, causing the stent 1100 to assume a less oval/more circular shape of the expanded form 1107, where the major axis 26b may be shorter than it was prior to the increase of blood pressure and/or the blood vessel 11 may have a larger cross-sectional area than in the more oval shape of FIG. 7.

While the struts forming the stent 1100 may be at least partially flexible and/or deformable to enable changing cross-sectional area of the stent 1100, the struts may be at least partially resistant to changes in shape. For example, if the stent 1100 is too responsive (e.g., too flexible), the stent 1100 may not sufficiently resist blood pressure increases during systole and/or can change shape prematurely. The struts may thus be configured to bend and/or change shape in response to pressure exceeding a threshold amount. In some examples, the struts may be thickened, however thickening of the struts may cause increased stress on the material of the struts as the struts bend.

The middle portion of the stent 1100 may be configured to extend at least partially across the lumen 1110 of the stent 1100 to form supporting arms and/or chords for the stent 1100. As a result, the middle portion can provide increased resistance to circularizing and/or can increase a force to pull the stent 1100 back to the relaxed form 1105 following systole.

At least part of the middle portion (e.g., the support arms 1120) may be formed by tension springs and/or struts having more curved shapes to provide increased spring behavior for the support arms extending across the lumen 1110 of the stent 1100. The tension springs of the support arms can be tuned (e.g., by their geometry and/or using thermal treatment of the struts) to allow the support arms to have spring behavior at least partially independently of the tubular geometry of the stent 1100. In some examples, the support arms may and/or other struts of the stent 1100 may be at least partially composed of one or more shape memory alloys (e.g., Nitinol) to allow the support arms to have relatively high resistance until a threshold level of blood pressure and/or to allow for movement from the relaxed form 1105 to the expanded form 1107 when the threshold level of blood pressure is reached. For example, the stent 1100 may be configured to move from the relaxed form 1105 to the expanded form 1107 at a given point during systole and/or not immediately at the beginning of systole. For example, the support arms may be tuned such that martensitic stress is reached at the threshold blood pressure.

The oval shape of the stent 1100 at the relaxed form 1105 may provide for a relatively high cross-sectional change along the minor axis of the stent 1100 between the relaxed form 1105 and the expanded form 1107. Most of the volume change of the stent 1100 may be applied above diastolic pressure and/or not immediately at the beginning of systole. In some examples, the middle portion of the stent may comprise one or more biphasic springs.

The middle portion may have a generally hexagonal shape, as illustrated in FIG. 11. For example, the middle portion may extend at least partially along the first major side 1102a and/or second major side 1102b and/or may form sets of two support arms 1120 at either end of the middle portion. The middle portion may comprise a first support arm 1120a and/or a second support arm 1120b configured to extend to and/or form a point at or near the first minor side 1104a of the stent 1100. For example, the point formed by the first support arm 1120a and the second support arm 1120b may be approximately in line with and/or coplanar with the first minor side 1104a formed by the first end portion and/or second end portion of the stent 1100. The middle portion may similarly comprise a set of two support arms 1120 forming a point at or in line with the second minor side 1104b, as shown in FIG. 11. As a result, the middle portion may have a width that is approximately equal to the widths of the first end portion and/or the second end portion of the stent 1100.

The first support arm 1120a and second support arm 1120b may be

interconnected and/or may extend into each other. In some examples, a joint between the first support arm 1120a and the second support arm 1120b at the first minor side 1104a of the stent may be connected to and/or extend into rounded sides of the first end portion and/or second end portion of the stent 1100 at the first minor side 1104a. The first support arm 1120a and/or second support arm 1120b may be generally angled and/or may extend from the first major side 1102a and/or second major side 1102b, respectively, at an approximately 45-degree angle and/or may be angled towards each other.

Migration of stents and/or other devices according to the invention can be prevented using various methods, elements, and combinations thereof. For example, endothelialization of the stent wall and/or projecting barbs can be used to secure devices to the blood vessel wall. In addition to such elements/techniques and/or in lieu of such elements/techniques, devices of the invention may include anchors adapted to extend into branching blood vessels/structures, which can serve to anchor the device in the main blood vessel in which the main stent body is deployed. For example, a device may comprise a main stent body (such as any of the stent assemblies discussed previously in this application) adapted to be deployed in a main blood vessel to provide compliance thereto, such as where the main stent body is adapted to change from a smaller cross-sectional area to a larger cross-sectional area. The device may further have one or more anchors extending in a non-parallel fashion from the main stent body. The particular anchors may each comprise an anchor stent body adapted to be deployed (such as via radial expansion) into contact with tissue (e.g., wall tissue) of a secondary blood vessel or other vascular structure which can branch off and/or otherwise extend generally sideways from the main blood vessel. Note that multiple anchors and/or stents may be used and/or can extend from different locations along the length and/or radial perimeter of the main stent body, depending on the particular application. One or more anchors may preferably be positioned at locations which can align with secondary blood vessels/structures as they branch off/extend from the main blood vessel. In some examples, the anchors may be self-expanding or plastically deformable (such as via balloon expansion). The anchors may, once expanded or otherwise deployed into contact with the tissue of the secondary blood vessel/structure, form generally circular lumens, oval lumens, peanut-shaped lumens, etc., and/or may be adapted to maintain a generally constant cross-sectional area and/or may be adapted to change from smaller cross-sectional area to larger cross-sectional area configurations responsive to blood flow.

In accordance with one or more implementations of the present disclosure, a stent comprises a network of elastically deformable struts forming a first end portion, a second end portion, and a middle portion situated between the first end portion and the second end portion and comprising one or more support arms extending at least partially across a lumen formed by the first end portion and the second end portion.

The first end portion may be configured to contact a blood vessel wall of a blood vessel. In some examples, the network of elastically deformable struts is configured to radially expand from a first configuration to a second configuration within the blood vessel into direct contact with the blood vessel wall, the first configuration defining a first major dimension, a first minor dimension, a first cross-sectional area, a first cross-sectional shape, and a first perimeter of the stent wall, and the second configuration defining a second major dimension, a second minor dimension that is greater than the first minor dimension, a second cross-sectional area that is greater than the first cross-sectional area, and the first perimeter of the stent wall.

In some examples, the first major dimension is greater than the second major dimension. The first minor dimension may be smaller than the second minor dimension.

The first cross-sectional shape may comprise an oval shape. In some examples, the blood vessel is an aorta, and wherein the first perimeter approximates or exceeds a perimeter of the aorta.

In some examples, the one or more support arms comprise tension springs configured to increase in length. The first end portion may be generally oval shaped and a length of the first end portion along a major axis may be greater than a width of the first end portion along a minor axis.

The first end portion may be configured to deform in response to blood flow such that the length of the first end portion at least partially decreases and the width of the first end portion at least partially increases. In some examples, the second end portion is generally oval shaped and a length of the second end portion along a major axis is greater than a width of the second end portion along a minor axis.

In some examples, the second end portion is configured to deform in response to blood flow such that the length of the second end portion at least partially decreases and the width of the second end portion at least partially increases. The middle portion may be generally oval shaped.

The middle portion may be generally rectangle shaped. In some examples, a length of the middle portion is less than the length of the first end portion and a width of the middle portion is approximately equal to the width of the first end portion.

In some examples, the middle portion comprises two support arms extending along the minor axis and two sides extending along the major axis. Each of the two sides of the middle portion may be coplanar with sides of the first end portion and the second end portion.

The length of the middle portion may be approximately half the length of the first portion. In some examples, the length of the middle portion is approximately seventy-five percent the length of the first portion.

In some examples, the one or more support arms are configured to expand in response to blood pressure increasing above a threshold amount.

The first portion may form generally rounded ends extending generally along a minor axis of the first portion. In some examples, the one or more support arms comprise sets of two angled support arms at either end of the middle portion that join at a point that is coplanar with the rounded ends of the first portion.

In some examples, the middle portion has a generally hexagonal shape.

Some implementations of the present disclosure relate to a system for providing compliance to a native blood vessel. The system comprises a catheter comprising a catheter distal portion configured to be percutaneously advanced within a patient's vasculature to the native blood vessel and a stent releasably secured to the catheter distal portion and forming a lumen extending between a first opening and a second opening of the stent, a first end portion having a first width, a second end portion having a width approximately equal to the first width, a middle portion having a width that is less than the first width, and one or more support arms extending from the middle portion at least partially across the lumen.

In some examples, the first end portion is configured to secure to a blood vessel wall of a blood vessel. In some examples, the stent is configured to radially expand from a first configuration to a second configuration within the blood vessel into direct contact with the blood vessel wall, the first configuration defining a first major dimension, a first minor dimension, a first cross-sectional area, a first cross-sectional shape, and a first perimeter of the stent wall, and the second configuration defining a second major dimension, a second minor dimension that is greater than the first minor dimension, a second cross-sectional area that is greater than the first cross-sectional area, and the first perimeter of the stent wall.

The first major dimension may be greater than the second major dimension. In some examples, the first cross-sectional shape comprises an oval shape.

In some examples, the blood vessel is an aorta, and wherein the first perimeter approximates or exceeds a perimeter of the aorta. The one or more support arms may comprise tension springs configured to increase in length.

The first end portion may be generally oval shaped and a length of the first end portion along a major axis is greater than a width of the first end portion along a minor axis. In some examples, the first end portion is configured to deform in response to blood flow such that the length of the first end portion at least partially decreases and the width of the first end portion at least partially increases.

In some examples, the second end portion is generally oval shaped and a length of the second end portion along a major axis is greater than a width of the second end portion along a minor axis. The second end portion may be configured to deform in response to blood flow such that the length of the second end portion at least partially decreases and the width of the second end portion at least partially increases.

The middle portion may be generally oval shaped. In some examples, the middle portion is generally rectangle shaped.

In some examples, a length of the middle portion is less than the length of the first end portion and a width of the middle portion is approximately equal to the width of the first end portion. The middle portion may comprise two support arms extending along the minor axis and two sides extending along the major axis.

Each of the two sides of the middle portion may be coplanar with sides of the first end portion and the second end portion. In some examples, the length of the middle portion is approximately half the length of the first portion.

The length of the middle portion may be approximately seventy-five percent the length of the first portion.

In some examples, the one or more support arms are configured to expand in response to blood flow changes. The first portion may form generally rounded ends extending generally along a minor axis of the first portion.

The one or more support arms may comprise sets of two angled support arms at either end of the middle portion that join at a point that is coplanar with the rounded ends of the first portion. In some examples, the middle portion has a generally hexagonal shape.

Depending on the example, certain acts, events, or functions of any of the processes or algorithms described herein can be performed in a different sequence, may be added, merged, or left out altogether. Thus, in certain examples, not all described acts or events are necessary for the practice of the processes.

### CERTAIN EXAMPLES

Example 1: A system for providing compliance to a native blood vessel, the system comprising: a catheter comprising a catheter distal portion configured to be percutaneously advanced within a patient's vasculature to the native blood vessel; and a stent releasably secured to the catheter distal portion and forming: a lumen extending between a first opening and a second opening of the stent; a first end portion having a first width; a second end portion having a width approximately equal to the first width; a middle portion having a width that is less than the first width; and one or more support arms extending from the middle portion at least partially across the lumen.

Example 2: The system of example 1, wherein the first end portion is configured to secure to a blood vessel wall of a blood vessel.

Example 3: The system of example 2, wherein the stent is configured to radially expand from a first configuration to a second configuration within the blood vessel into direct contact with the blood vessel wall, the first configuration defining a first major dimension, a first minor dimension, a first cross-sectional area, a first cross-sectional shape, and a first perimeter, and the second configuration defining a second major dimension, a second minor dimension that is greater than the first minor dimension, a second cross-sectional area that is greater than the first cross-sectional area, and the first perimeter.

Example 4: The system of example 3, wherein the first major dimension is greater than the second major dimension.

Example 5: The system of example 3 or example 4, wherein the first cross-sectional shape comprises an oval shape.

Example 6: The system of any of examples 3-5, wherein the blood vessel is an aorta, and wherein the first perimeter approximates or exceeds a perimeter of the aorta.

Example 7: The system of any of examples 1-6, wherein the one or more support arms comprise tension springs configured to increase in length.

Example 8: The system of any of examples 1-6, wherein the first end portion is generally oval shaped and a length of the first end portion along a major axis is greater than a width of the first end portion along a minor axis.

Example 9: The system of example 8, wherein the first end portion is configured to deform in response to blood flow such that the length of the first end portion at least partially decreases and the width of the first end portion at least partially increases.

Example 10: The system of example 8 or example 9, wherein the second end portion is generally oval shaped and a length of the second end portion along a major axis is greater than a width of the second end portion along a minor axis.

Example 11: The system of example 10, wherein the second end portion is configured to deform in response to blood flow such that the length of the second end portion at least partially decreases and the width of the second end portion at least partially increases.

Example 12: The system of any of examples 8-11, wherein the middle portion is generally oval shaped.

Example 13: The system of any of examples 8-12, wherein the middle portion is generally rectangle shaped.

Example 14: The system of any of examples 8-13, wherein a length of the

middle portion is less than the length of the first end portion and a width of the middle portion is approximately equal to the width of the first end portion.

Example 15: The system of example 14, wherein the middle portion comprises two support arms extending along the minor axis and two sides extending along the major axis.

Example 16: The system of example 15, wherein each of the two sides of the middle portion is coplanar with sides of the first end portion and the second end portion.

Example 17: The system of any of examples 8-16, wherein the length of the middle portion is approximately half the length of the first end portion.

Example 18: The system of any of examples 8-17, wherein the length of the middle portion is approximately seventy-five percent the length of the first end portion.

Example 19: The system of any of examples 1-18, wherein the one or more support arms are configured to expand in response to blood flow changes.

Example 20: The system of any of examples 1-19, wherein the first end portion forms generally rounded ends extending generally along a minor axis of the first end portion.

Example 21: The system of example 20, wherein the one or more support arms comprise sets of two angled support arms at either end of the middle portion that join at a point that is coplanar with the rounded ends of the first end portion.

Example 22: The system of any of examples 1-21, wherein the middle portion has a generally hexagonal shape.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y, and at least one of Z to each be present.

It should be appreciated that in the above description of examples, various features are sometimes grouped together in a single example, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

Each element of each example and its respective elements disclosed herein can be used with any other example and its respective elements disclosed herein. All dimensions listed are by way of example, and devices according to the invention may have dimensions outside those specific values and ranges. The dimensions and shape of the device and its elements depend on the particular application. Unless otherwise noted, all technical and scientific terms used herein have the same meaning as commonly understood by one of ordinary skill in the art to which this disclosure belongs. In order to facilitate review of the various examples of the disclosure, the following explanation of terms is provided:

The singular terms “a”, “an”, and “the” include plural referents unless context clearly indicates otherwise. The term “or” refers to a single element of stated alternative elements or a combination of two or more elements, unless context clearly indicates otherwise. The term “includes” means “comprises.” For example, a device that includes or comprises A and B contains A and B, but may optionally contain C or other components other than A and B. Moreover, a device that includes or comprises A or B may contain A or B or A and B, and optionally one or more other components, such as C.

The term “subject” refers to both human and other animal subjects. In certain examples, the subject is a human or other mammal, such as a primate, cat, dog, cow, horse, rodent, sheep, goat, or pig. In a particular example, the subject is a human patient.

Although methods and materials similar or equivalent to those described herein can be used in the practice or testing of the present disclosure, suitable methods and materials are described below. In case of conflict, the present specification, including terms, will control. In addition, the materials, methods, and examples are illustrative only and not intended to be limiting.

In view of the many possible examples to which the principles of the disclosed invention may be applied, it should be recognized that the illustrated examples are only examples of the invention and should not be taken as limiting the scope of the invention. Rather. the scope of the invention is defined by the following claims. We therefore claim as our invention all that comes within the scope and spirit of these claims.

What is claimed is:

1. A collapsible and expandable stent sized for placement in an aorta, the stent comprising:
a network of elastically deformable struts forming a scaffold including multiple portions, at least some of the portions having a biased substantially oval cross-section with major and minor dimensions, wherein the at least some of the portions of the scaffold contract along a major dimension and expand in a minor dimension with increased blood pressure in the aorta, the scaffold forming:
a first end portion having the biased substantially oval cross-section including a first central diameter in the major dimension and a second central diameter in the minor dimension, wherein the first central diameter is greater than the second central diameter;
a second end portion having the biased substantially oval cross-section including the first central diameter in the major dimension and the second central diameter in the minor dimension; and
a middle portion situated between the first end portion and the second end portion, the middle portion having a third central diameter in the major dimension and the second central diameter in the minor dimension, the third central diameter being less than the first central diameter and extending between first and second tension springs that resist circularization of the first and second end portions.

2. The stent of claim 1, wherein the first end portion and the second end portion expand in the minor dimension into direct contact with a wall of the aorta.

3. The stent of claim 2, wherein the first end portion and the second end portion contract in the major dimension as the first end portion and the second end portion expand in the minor dimension into direct contact with the wall of the aorta.

4. The stent of claim 1, wherein the first and second tension springs expand in response to blood pressure increasing above a threshold amount.

5. The stent of claim 1, wherein the at least some of the portions of the scaffold expand along the major dimension and contract in the minor dimension with deccreased blood pressure in the aorta.

6. The stent of claim 1, wherein the middle portion has a different shape than the first end portion.

7. The stent of claim 1, wherein the third central diameter is approximately half the first central diameter.

8. The stent of claim 1, wherein the third central diameter is approximately seventy-five percent of the first central diameter.

9. The stent of claim 1, wherein the first end portion forms rounded sides and wherein the first and second tension springs join at a point that is coplanar with the rounded sides of the first end portion.

10. The stent of claim 1, further comprising one or more sensors coupled to the network of elastically deformable struts.

11. The stent of claim 1, further comprising a covering extending across openings of the scaffold and creating a sealing between the scaffold and the aorta.

12. A stent for adding compliance to a blood vessel, the stent comprising:
a network of elastically deformable struts forming a scaffold including multiple portions, at least some of the portions having a biased non-circular cross-section with major and minor dimensions, wherein the at least some of the portions of the scaffold contract along a major dimension and expand in a minor dimension with increased blood pressure in the blood vessel, the scaffold forming:
a first end portion having the biased non-circular cross-section including a first central diameter in the major dimension and a second central diameter in the minor dimension, wherein the first central diameter is greater than the second central diameter;
a second end portion having the biased non-circular cross-section including the first central diameter in the major dimension and the second central diameter in the minor dimension; and
a middle portion situated between the first end portion and the second end portion and comprising one or more support arms extending at least partially across a lumen formed by the first end portion and the second end portion, wherein a shape of the middle portion is different than shapes of the first end portion and second end portion.

13. The stent of claim 12, wherein the first end portion is oval-shaped.

14. The stent of claim 13, wherein the middle portion has a hexagonal shape.

15. The stent of claim 13, wherein the middle portion has an oval shape.

16. The stent of claim 13, wherein the middle portion has a rectangular shape.

17. The stent of claim 12, further comprising a covering extending across openings of the scaffold and creating a sealing between the scaffold and the blood vessel.

18. A stent for adding compliance to a blood vessel for improving blood circulation, the stent comprising:
a network of elastically deformable struts forming a tubular scaffold including multiple portions, at least some of the portions having a biased non-circular cross-section with major and minor dimensions, wherein the at least some of the portions of the scaffold contract along a major dimension and expand in a minor dimension with increased blood pressure in the blood vessel, the scaffold forming:
a first end portion having the biased non-circular cross-section including a first central diameter in the major dimension and a second central diameter in the minor dimension, wherein the first central diameter is greater than the second central diameter;
a second end portion having the biased non-circular cross-section including the first central diameter in the major dimension and the second central diameter in the minor dimension; and
a middle portion situated between the first end portion and the second end portion and comprising two or more support arms forming sides of the middle portion, the two or more support arms extending at least partially across a lumen formed by the first end portion and the second end portion;
wherein elastic deformation of the stent emulates the compliance of a healthy blood vessel during cyclic changes in luminal blood pressure, thereby improving circulation in a patient.

19. The stent of claim 18, wherein the middle portion has a third central diameter along the major dimension and between the two or more support arms, the third central diameter being less than the first central diameter.

20. The stent of claim 18, further comprising a covering extending across openings of the scaffold and creating a sealing between the scaffold and the blood vessel.
